(19) United States
(12) Patent Application Publication
(10) Pub. No.: US 2004/0157274 A1
(43) Pub. Date: Aug. 12, 2004

(54) Method for monitoring the rate of t-cells recently emigrated from the thymus

(76) Inventor: Richard W. Olaussen, Nesodden (NO)

Correspondence Address: HARNESS, DICKEY & PIERCE, P.L.C., P.O. BOX 828, BLOOMFIELD HILLS, MI, 48303, US

(21) Appl. No.: 10/482,262

(22) PCT Filed: Jun. 19, 2002
(86) PCT No.: PCT/NO02/00218

(30) Foreign Application Priority Data
Jun. 25, 2001 (NO) 20013192

(51) Int. Cl. G01N 33/53; G01N 33/567

(52) U.S. Cl. 435/7.21

(57) ABSTRACT

The chemokine receptor CCR9 is reported to be predominantly expressed by thymocytes as well as by circulating gut-homing and resident T cells in the small intestinal mucosa. Its ligand TECK (thymus-expressed chemokine) is produced by thymic and small intestinal epithelium. Here we report that the relative fraction of circulating CCR9⁺ naive T cells (mostly CD4+) declines with age, from approximately 15% of all T cells at birth to around 1% in adults. The proportion of CCR9⁺ T cells negative for the classical gut homing marker α4β7, was much higher in childhood than in adults. Therefore, circulating CD3⁺CD45RA⁺CCR9⁺ cells have most likely left the thymus quite recently. Establishing a phenotypic marker for recent thymic emigrants may provide a powerful tool in the clinical assessment and follow-up after hematopoietic stem cell transplantation and during antiretroviral treatment of HIV-infected patients.

### INTRODUCTION

The present invention relates to an in vitro method for monitoring the rate of T-cells, and a kit comprising antibodies for monitoring T-cells.

The human G protein-coupled receptor GPR-9-6/CCR9 was described by Zaballos et al. ¹ as the receptor for the thymus-expressed chemokine (TECK). TECK is apparently produced only by thymic dendritic² and epithelial³ cells, and by enterocytes of the small intestine^{3,4}. CCR9 expression on circulating memory cells is reported to define a gut-homing subset⁵, in agreement with the finding that most T cells in the small intestinal epithelium and lamina propria express CCR9^{4,6}. Little is known about the regulation of CCR9 with age. In mice, CD8⁺ thymocytes, as well as CD8⁺ cells in peripheral lymphoid organs, constitute, a CCR9⁺CD69^{low}CD62^{high} subset that decreasingly migrates towards TECK with increasing age⁷. In humans, recently emigrated thymocytes can be identified among circulating cells by TCR-rearrangement excision circles in episomal DNA⁸, but no applicable phenotypic marker has to our knowledge been described. Here, we report that most CCR9⁺ T cells in children are of the naive (CD45RA⁺) phenotype. This population is much lower in adults and markedly reduced in recently thymectomized children. Therefore, we postulate that circulating CD45RA⁺CCR9⁺ T cells mainly represent recent thymic emigrants. The method of the present invention is an excellent tool in determining the extent of immunefailure in different diseases and monitoring the effect of treatment.

Study Design

Peripheral blood from pediatric patients (n=20, 4 months-18 years ) and adult healthy volunteers (n=9, 27-59 years) was used. The study was approved by the regional ethical committee, and written informed consent was obtained from parents (or children more that 12 years old). Blood was collected for this study only when venous puncture was carried out for diagnostic or follow-up purposes of their ailments. The patients did not receive immunosuppressive treatment and were not afflicted with known immune dysfunctions. E.g., constipation, neurosis, epilepsy, migraine or temporomandibular joint ankylosis. Blood was also obtained from five children 1 week (age at investigation: 5 years), 6 weeks (age: 4 months), 11 weeks (age: 3 months), 3 years (age: 4 years), and 4 years (age: 4 years) after thymectomy which was carried out in connection with cardiac surgery.

Heparinized blood was separated with Lymphoprep (Nycomed Pharma, Oslo, Norway) and subjected to double- or triple-color immunofluorescence staining with two or three incubation steps. The following antibody reagents were used: mouse anti-CD3-FITC (fluorescein isothiocyanate), mouse anti-CD4 (IgG1; SK3 and SK4), mouse anti-CD8 (IgG1, SK1), mouse anti-CD45RA-FITC, mouse anti-CD3-PerCP (peridinin chlorophyll) (all from Becton Dickinson, San Jose, Calif.), mouse anti-CCR9 (LS 129 3C3, IgG2b; courtesy of Dr. Paul Ponath, LeukoSite Inc, Cambridge, Mass.), mouse anti-α4β7 (Act. 1, IgGl, courtesy of Dr. Andrew I. Lazarovits, Ontario, Canada), goat anti-mouse IgG2b-PE (phycoerythrin), (Southern Biotechnology Associates, Birmingham, Ala.) and goat anti-mouse Ig1 PE/CyS (PE/indodicarbocyanide) (Caltag Laboratories, Burlingame, Calif.). Controls were isotype- and concentration-matched antibodies of irrelevant specificities. Acquisition was carried out on a FACScan or FACS Vantage SE Flow cytometer equipped with the Cell Quest analysis programme (Becton Dickinson, San Jose, Calif.). SPSS for Windows, release 9.0.1 (Chicago, Ill.) was used for statistical analyses of the compiled flow-cytometric data.

### FIGURE LEGENDS

FIG. 1

Variation of CCR9⁺ naive T cells in relation to age. The upper right quadrant of diagram A through G represents CCR9⁺ naive T cells as defined by the CD45RA marker. A-F: After 1 year of age, the proportion of this subset shows a decrease that levels out in adulthood. The same subset is strikingly reduced in a thymectomized patient (IC). G: The CCR9⁺ naive T cell population was quite low in a 59-year-old blood donor compared with that of a 51-year-old subject. This accords with a steeper decrease of naive T cells from about 60 years of age and onwards as reported by others⁸. H-I: A higher percentage of CCR9⁺ T cells negative for the gut homing marker α4β7 is seen in a child compared with that of an adult.

FIG. 2

Compiled flow-cytometric data in relation to age of study subjects. A: percentage of CCR9⁺ cells among all T cells decreases with age. B: When a natural log transformation of the same data is plotted versus age, a linear relationship appears. C, D: Comparable data for CCR9⁺ CD45RA⁺ T cells.

FIG. 3

Compiled flow-cytometric data for CCR9+naïve T cells (A) and CCR9+a4b7-T cells (B) in normal (o) and thymectomized (▴) subjects in relation to age. Data from FIG. 2C for ages under 10 years old and, in addition, data from the thymectomized patients. The time from thymectomy was 6 and 11 weeks, respectively, for the patients located in the lower left corner, and 4 years in the 4 year-old-patient with the lowest value. The respective times for the other 4-year-old and the 5-year-old patient were 3 years and 1 week.

### RESULTS AND DISCUSSION

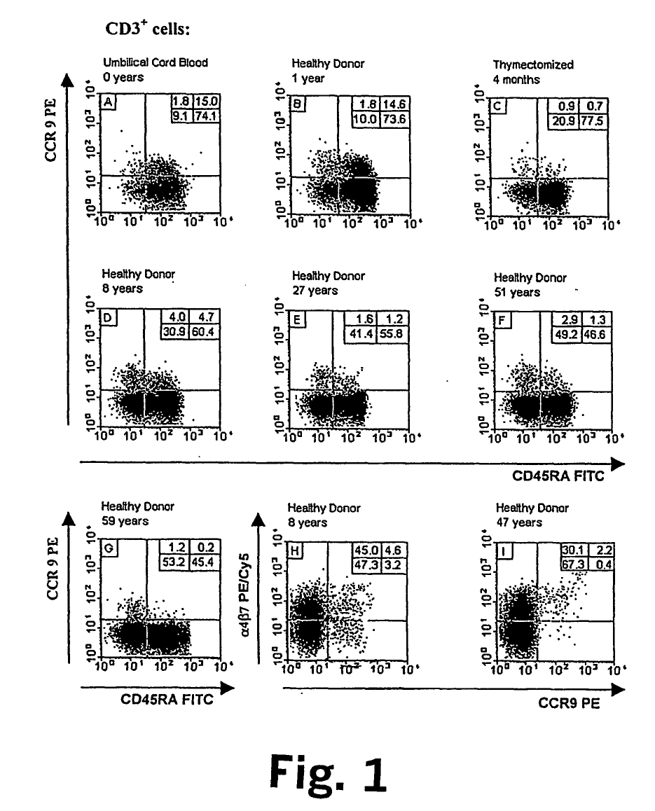

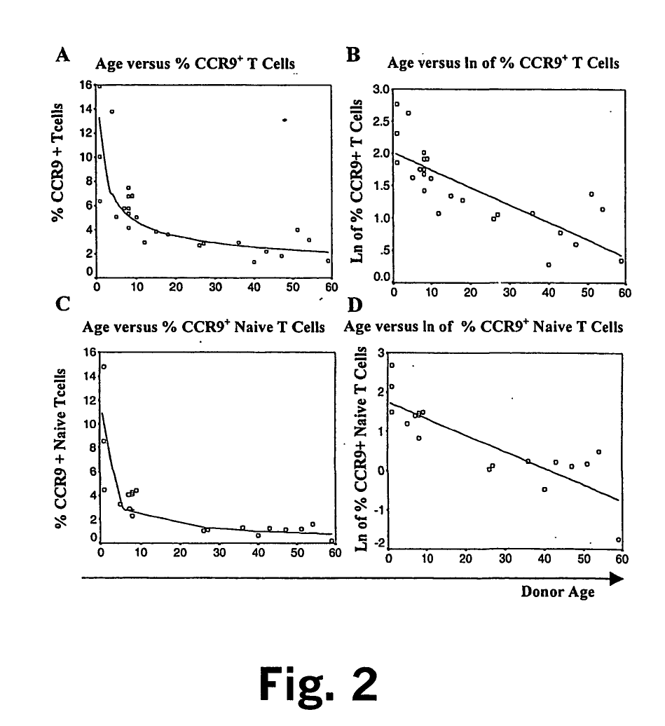

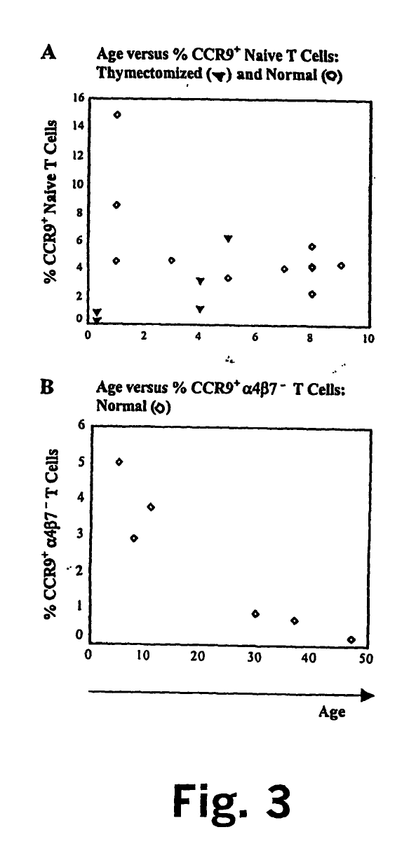

FIG. 1 shows expression of CD45RA and CCR9 on CD3⁺ gated cells (Figure 1A-1G). Except for data from a thymectomized patient (FIG. 1C), CD45RA⁺CCR9⁺CD3⁺ cells constituted a relatively large fraction (˜15%) during the first year of life, declining to ˜5% by adolescence and to ˜1% in adults. The age-dependent decrease of CD3⁺CD45RA⁺CCR9⁺ cells parallels the age-dependent thymic involution⁹ (see below), and circulating CCR9⁺ naive T cells might therefore represent recent thymic emigrants. According to Campbell et al¹⁰, the most mature medullary thymocytes migrate poorly towards TECK, although the CCR9 expression of those cells was not examined. No age-dependent relationship was observed for the CD45RA⁻CCR9⁺ T cell population. The mean fluorescence intensity was lower for CD45RA⁺ cells than for CD45RA⁻ cells (data not shown), in agreement with data from Zabel et al.⁵, and the CD4:CD8 ratio among the naive CCR9⁺ population was 2.5:1 (data not shown). This last result differed from the reported CD8⁺ predominance in murine CCR9-expressing cells found in peripheral lymph nodes⁷.

FIG. 1C displays data from a 3-month-old patient 11 weeks after thymectomy; a marked reduction of the CD45RA⁺CCR9⁺ T cell population was observed compared with that present in umilical cord blood (FIG. 1A) and the 1-year-old donor (Figure 1B); another 4-month-old patient showed the same marked reduction 6 weeks after thymectomy; and in a 4-year-old patient thymectomized a few days after birth the population was reduced by 75% relative to age-matched controls -(data not shown). Conversely, no decrease of the CCR9⁺ naive T cell population was seen 1 week after thymectomy in a 5-year-old patient, and only a tendency towards reduction was seen 3 years after thymectomy in another 4-year-old patient (3.1% as compared to a median of 4.1% in age matched controls). Patient age, extent of (subtotal or total), and time after thymectomy could all contribute to the observed results. Remnants of thymic tissue might be functional and over time exert a compensatory regeneration of thymocytes. Thus, TCR-rearrangement-excision-circle studies of thymectomized patients more than 3 years after thymectomy showed that new T cells were produced although at a slightly reduced level⁸.

Figure 1H and Figure 1I show that the classical intestinal homing receptor a4b7 was absent on approximately 50% of the CD3+CCR9+ cells in an 8-year-old donor but occurred on most CD3+CCR9+cells of a 47 year-old donor. Similar data are compiled in FIG. 3B for a total of three children and three adults. CCR9+ on circulating T-cells has been associated with homing to the small intestine [4,5]. However, according to our results described above, the CCR9+a4b7-cells could rather be derived from the thymus. A.two-fold reduction was seen for the CCR9+a4b7+ T-cells as opposed to a four-fold reduction among the CCR9+a4b7− T-cells from 5 to 47 years (data not shown). This suggested a link between the two cell populations but would only partially account for the findings with respect to CCR9+a4b7− cells and ageing. In fact, CCR9+a4b7− T-cells could also be recent thymic emigrants. Also notable in this context, the relative proportion of CCR9-expressing T cells decreased rapidly over the first 10 years of life, whereafter there was a more gradual decline (FIG. 2A). The adapted curve assumed the shape of a power function of the form y=ax^{b}, and others have used this model function to describe the involution of the thymus cortex⁹. The natural log-transformed percentage variable had a normal distribution; when this, variable was plotted against age (FIG. 2B), a significant inverse correlation appeared (Pearsons r=−0.79, P<0.01). FIG. 2C shows that the rapid initial decline was confined to the CD45RA⁺population of CCR9⁺ T cells (based on 18 donors); and a plot of the natural log-transformed percentage variable versus age (FIG. 2D) again showed a significant negative correlation (r=−0.86, P<0.01).

In conclusion, the inventors have observed a significant age-related decline of circulating CCR9⁺ T cells restricted to CD45RA⁺ cells. The CD3⁺CCR9^{+CD}45RA⁺ population was consistently reduced dramatically after thymectomy, until after a variable period it again appeared to be increased, probably because of a compensatory mechanism. We therefore suggest that this naive T cell population represents recent thymic emigrants. The method of the present invention provides a convenient tool for monitoring the rate of T-cells recently emigrated from the thymus (RTEs) by e.g. monitoring responses to antiretroviral treatment in HIV-infected patients⁸ or assess the extent of infection in said patients or to assess the regeneration of T cells after transplantation e.g. in hematopoietic stem cell transplantation¹¹ or in patients suffering from autoimmune diseases. The effect on thymic function of cancer patients and in cancer related suppression after chemotherapy or irradiation as well as T cell reconstitution, can also be studied and easily monitored¹² by the method of the present invention. The present invention relates also to a kit comprising at least one type of antibody, which can be conjugated to e.g. fluorochrome for monitoring recently emigrated T-cells. The method of the present invention has also a range of application in scientific work e.g. by studying the relation between cancer and RTEs deficiency, the relation between age and the share of RTEs or in the field of autoimmune diseases.

References

1. Zaballos A, Gutierrez J, Varona R, Ardavin C, Marquez G. Cutting edge: identification of the orphan chemokine receptor GPR-9-6 as CCR9, the receptor for the chemokine TECK. J. Immunol. 1999; 162:5671-5675.

2. Vicari A P, Figueroa D J, Hedrick J A, et al. TECK: a novel CC chemokine specifically expressed by thymic dendritic cells and potentially involved in T cell development. Immunity. 1997;7:291-301.

3. Wurbel M A, Philippe J M, Nguyen C, et al. The chemokine TECK is expressed by thymic and intestinal epithelial cells and attracts double- and single-positive thymocytes expressing the TECK receptor CCR9. Eur.J.Irnmunol. 2000;30:262-271.

4. Kunkel E J, Campbell J J, Haraldsen G, et al. Lymphocyte CC chemokine receptor 9 and epithelial thymus-expressed chemokine (TECK) expression distinguish the small intestinal immune compartment: Epithelial expression of tissue-specific chemokines as an organizing principle in regional immunity. J.Exp.Med. 2001;192:761-768.

5. Zabel B A, Agace W W, Campbell J J, et al. Human G protein-coupled receptor GPR-9-6/CC chemokine receptor 9 is selectively expressed on intestinal homing T lymphocytes, mucosal lymphocytes, and thymocytes and is required for thymus-expressed chemokine-mediated chemotaxis. J.Exp.Med. 1999;190:1241-1256.

6. Papadakis K A, Prehn J, Nelson V, et al. The role of thymus-expressed chemokine and its receptor CCR9 on lymphocytes in the regional specialization of the mucosal immune system. J.Immunol. 2000;165:5069-5076.

7. Carramolino L, Zaballos A, Kremer L, et al. Expression of CCR9 beta-chemokine receptor is modulated in thymocyte differentiation and is selectively maintained in CD8(+) T cells from secondary lymphoid organs. Blood 2001;97:850-857.

8. Douek D C, McFarland R D, Keiser P H, et al. Changes in thymic function with age and during the treatment of HIV infection. Nature 1998;396:690-695.

9. Steinmann G G. Changes in the human thymus during aging. Curr.Top.Pathol. 1986;75:43-88.

10. Campbell J J, Pan J, Butcher E C. Cutting edge: developmental switches in chemokine responses during T cell maturation. J.Immunol. 1099;163:2353-2357.

11. Douek D C, Vescio R A, Betts M R, et al. Assessment of thymic output in adults after haematopoietic stem-cell transplantation and prediction of T-cell reconstitution. Lancet 2000;355:1875-1881.

12. Mackall C L, Fleisher T A, Brown M R, et al. Age, thymopoiesis, and CD4+ T-lymphocyte regeneration after intensive chemotherapy. N.Engl.J.Med. 1995;332:143-149.

1. A method for monitoring T-cells, characterised in that specifically binding molecules are used to detect newly formed T-cells in that a specific binding between said newly formed T-cells and said specifically binding molecules is obtained by contacting said newly formed T-cells with a combination of specifically binding molecules, and wherein detection of this or these specific bindings is carried out instrumentally.

2. The method according to claim 1, characterised in that said T-cells are human T-cells.

3. The method according to claims 1-2, characterised in that said T-cells are from blood and/or bone marrow or other tissue.

4. The method according to claims 1-3, characterised in that said specifically binding molecules are antibodies and/or molecules derived from antibodies.

5. The method according to claims 1-4, characterised in that detection is carried out using a flow cytometer.

6. The method according to claims 1-5, characterised in that said specifically binding molecules are used against cell markers on said T-cells, wherein said specifically binding molecules are against T-cell markers selected from the group(s) comprising: CD3, CD2, CD7 and/or CD4 or CD8 in combination with said specifically binding molecules against the cell marker CCR9 and additional said specifically binding molecules against at least one of the cell markers CD45RA or α4β7.

7. The use of a combination of antibodies and/or other specifically binding molecules which provide a specific binding between newly formed T-cells and antibodies and/or other specifically binding molecules for monitoring T-cells.

8. A kit, characterised in that it comprises a selection of antibodies and/or specifically binding molecules capable of binding specifically to newly formed T-cells.

9. A kit according to claim 8, characterised in that it comprises a selection of said antibodies and/or other specifically binding molecules, wherein said antibodies and/or specifically binding molecules are against the cell markers according to claim 6.
